(19) United States
(12) Patent Application Publication
(10) Pub. No.: US 2025/0066471 A1
(43) Pub. Date: Feb. 27, 2025

(54) ANTI-ROBO4 HUMAN MONOCLONAL ANTIBODIES AND USES THEREOF FOR THE TREATMENT OF CANCER

(71) Applicants: INSTITUT NATIONAL DE LA SANTÉ ET DE LA RECHERCHE MÉDICALE, Paris (FR); UNIVERSITÉ DE MONTPELLIER, Montpellier (FR); INSTITUT RÉGIONAL DU CANCER DE MONTPELLIER, Montpellier (FR); UNIVERSITÉ CLAUDE BERNARD - LYON 1, Villeurbanne (FR)

(72) Inventors: Philippe CLEZARDIN, Lyon - Cedex 08 (FR); Bruno ROBERT, Montpellier Cedex 5 (FR); Pierre MARTINEAU, Montpellier Cedex 5 (FR); Bénédicte ECKEL, Chaponost (FR); Chantal DIAZ-LATOUD, Lyon Cedex 08 (FR); Lise CLEMENT-DEMANGE, Grenoble (FR); Myriam CHENTOUF, Montpellier Cedex 5 (FR); Margaux BERNARD, Paris (FR)

(21) Appl. No.: 18/729,000

(22) PCT Filed: Feb. 15, 2023
(86) PCT No.: PCT/EP2023/053725

§ 371 Date: Jul. 15, 2024

(30) Foreign Application Priority Data
Feb. 16, 2022 (EP) 22305173.1

(51) Int. Cl. C07K 16/28; A61P 35/00

(52) U.S. Cl. CPC C07K 16/2803; A61P 35/00; C07K 2317/21; C07K 2317/565

(57) ABSTRACT

There is an interest to develop anti-ROBO4 antibodies that can be suitable for the treatment of cancer. The inventors produced new human antibodies directed against ROBO4 were by a phage display strategy and the antibody selection was performed by using HEK and HEK stably expressing ROBO4 (HEK-ROBO4) cell lines. As a first step, a batch of 6 different antibodies named D3, H3, H9, E11, H11 and G12 was tested for their binding properties. The antibodies were then tested for their ability to inhibit the attachment of tumor cells to osteoblastic cells in monolayers. Finally the inventors showed that the anti-ROBO4 antibodies E11 and G12 inhibits significantly the formation of the tumor spheroid. The present invention thus relates to anti-ROBO4 human monoclonal antibodies and uses thereof for the treatment of cancer.

### FIELD OF THE INVENTION

The present invention is in the field of medicine, in particular oncology.

### BACKGROUND OF THE INVENTION

There are four members of the ROBO family (ROBO1, ROBO2, ROBO3 and ROBO4) in mammals. ROBO1, ROBO2, ROBO3 and their ligands (SLITs) have been initially shown as repelling axons from migrating to inappropriate locations during the assembly of the nervous system (5). ROBO1 and ROBO4 are also expressed in the vasculature. ROBO1 promotes endothelial cell motility in vitro and stimulates angiogenesis in vivo. In contrast, ROBO4 maintains vascular integrity by counteracting the stimulatory effects of VEGF on angiogenesis and vascular permeability.

Little is known about the role of ROBO4 in cancer. ROBO4 is a tumor angiogenesis marker in animal models of cancer. For example, the targeting of endothelial Robo4 is important for inhibiting tumor angiogenesis that, in turn, reduces tumor growth and lung metastasis in a murine model of breast cancer. However, ROBO4 expression is not restricted to endothelial cells. ROBO4 is also expressed in epithelial tumor cells in human breast, prostate, and ovarian cancers. The contribution of tumor ROBO4 to cancer and metastasis was also suggested (WO2014/072416).

There is thus an interest to develop anti-ROBO4 antibodies that can be suitable for the treatment of cancer. WO2008100805 and WO2013160879 describe anti-ROBO4 antibodies. Anti-ROBO4 antibodies are also commercially available (e.g. MAB24541 R&D System).

### SUMMARY OF THE INVENTION

The present invention is defined by the claims. In particular, the present invention relates to anti-ROBO4 human monoclonal antibodies and uses thereof for the treatment of cancer.

### DETAILED DESCRIPTION OF THE INVENTION

### Main Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention, a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “conservative sequence modifications” refers to amino acid modifications that do not significantly affect or alter the biologic function of the protein containing the amino acid sequence. Such conservative modifications include amino acid substitutions, additions and deletions. Modifications can be introduced into a protein by standard techniques known in the art, such as site-directed mutagenesis and PCR-mediated mutagenesis. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Other families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “polynucleotide sequence encoding an amino acid sequence” includes all nucleotide sequences that are degenerate versions of each other and that encode the same amino acid sequence. The phrase “polynucleotide sequence that encodes a protein or a RNA” may also include introns to the extent that the nucleotide sequence encoding the protein may in some version contain an intron(s).

As used herein, the term “ROBO4” has its general meaning in the art and refers to the cell surface transmembrane protein Roundabout 4. ROBO4 was first described as an axon guidance receptor protein, and its amino acid sequence and the gene sequence coding thereof are disclosed in GenBank ID [acc. no. AF361473]. An exemplary amino acid sequence for ROBO4 is represented by SEQ ID NO:1. As used herein, the term “ROBO4 92-kDa isoform” refers to the ROBO4 isoform having a molecular weight of 92-kDa as described in the EXAMPLE.

>sp|Q8WZ75|ROBO4_HUMAN Roundabout homolog 4 OS = Homo sapiens
OX = 9606 GN = ROBO4 PE = 1 SV = 1
SEQ ID NO: 1
MGSGGDSLLGGRGSLPLLLLLIMGGMAQDSPPQILVHPQDQLFQGPGPARMSCQASGQPP
PTIRWLLNGQPLSMVPPDPHHLLPDGTLLLLQPPARGHAHDGQALSTDLGVYTCEASNRL
GTAVSRGARLSVAVLREDFQIQPRDMVAVVGEQFTLECGPPWGHPEPTVSWWKDGKPLAL
QPGRHTVSGGSLLMARAEKSDEGTYMCVATNSAGHRESRAARVSIQEPQDYTEPVELLAV
RIQLENVTLLNPDPAEGPKPRPAVWLSWKVSGPAAPAQSYTALERTQTAPGGQGAPWAEE
LLAGWQSAELGGLHWGQDYEFKVRPSSGRARGPDSNVLLLRLPEKVPSAPPQEVTLKPGN
GTVFVSWVPPPAENHNGIIRGYQVWSLGNTSLPPANWTVVGEQTQLEIATHMPGSYCVQV
AAVTGAGAGEPSRPVCLLLEQAMERATQEPSEHGPWTLEQLRATLKRPEVIATCGVALWL
LLLGTAVCIHRRRRARVHLGPGLYRYTSEDAILKHRMDHSDSQWLADTWRSTSGSRDLSS
SSSLSSRLGADARDPLDCRRSLLSWDSRSPGVPLLPDTSTFYGSLIAELPSSTPARPSPQ
VPAVRRLPPQLAQLSSPCSSSDSLCSRRGLSSPRLSLAPAEAWKAKKKQELQHANSSPLL
RGSHSLELRACELGNRGSKNLSQSPGAVPQALVAWRALGPKLLSSSNELVTRHLPPAPLF
PHETPPTQSQQTQPPVAPQAPSSILLPAAPIPILSPCSPPSPQASSLSGPSPASSRLSSS
SLSSLGEDQDSVLTPEEVALCLELSEGEETPRNSVSPMPRAPSPPTTYGYISVPTASEFT
DMGRTGGGVGPKGGVLLCPPRPCLTPTPSEGSLANGWGSASEDNAASARASLVSSSDGSF
LADAHFARALAVAVDSFGFGLEPREADCVFIDASSPPSPRDEIFLTPNLSLPLWEWRPDW
LEDMEVSHTQRLGRGMPPWPPDSQISSQRSQLHCRMPKAGASPVDYS

As used herein the term “antibody” and “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

As used herein the term “human antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “Fc region” has its general meaning in the art and includes the polypeptides comprising the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term “effector cells” are leukocytes which express one or more FcRs and perform effector functions. The cells express at least FcγRI, FCTRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “epitope” refers to a specific arrangement of amino acids located on a protein or proteins to which an antibody binds. Epitopes often consist of a chemically active surface grouping of molecules such as amino acids or sugar side chains, and have specific three-dimensional structural characteristics as well as specific charge characteristics. Epitopes can be linear or conformational, i.e., involving two or more sequences of amino acids in various regions of the antigen that may not necessarily be contiguous.

As used herein, the term “neutralizing anti-ROBO4 monoclonal antibody” refers to an antibody to a monoclonal antibody having specificity for ROBO4 and that reduces at least one activity of ROBO4. In particular, the neutralizing anti-ROBO4 monoclonal antibody inhibits the formation of tumor spheroid. Said activities can be measured by any well-known method in the art and typically as described in EXAMPLE.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, “consisting essentially of”, with reference to a composition, means that the at least one antibody of the invention as described hereinabove is the only one therapeutic agent or agent with a biologic activity within said composition.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

### Antibodies of the Present Invention:

The first object of the present invention relates to an anti-ROBO4 human monoclonal antibody having:
a light chain comprising the complementarity determining regions CDR1L, CDR2L and CDR3L,
   the CDR1L having the amino acid sequence AGTSSDVGGY-X₁₁-X₁₂-VS (SEQ ID NO:2) wherein X₁₁ is Y or S and X₁₂ is G or Y
   the CDR2L having the amino acid sequence X₁-DS-X₄-RPS (SEQ ID NO:3) wherein X₁ is Y or S and X₄ is N or S
   and the CDR3L having the amino acid sequence SSYT-X₅-X₆-X₇-TRV (SEQ ID NO:4) wherein X₅ is S or N, X₆ is Y or S and X₇ is S or A and
a heavy comprising the complementarity determining regions CDR1H, CDR2H and CDR3H,
   the CDR1H having the amino acid sequence N-X₂-YMN (SEQ ID NO:5) wherein X₂ is Y or N,
   the CDR2H having the amino acid sequence X₁-I-X₃-GSSR-X₈-I-X₁₀-YADFVKG (SEQ ID NO:6) wherein X₁ is G or N, X₃ is S or A, X₈ is Y or G, X₁₀ is N or Y,
   and the CDR3H having the amino acid sequence S-X₂-Y-X₄-GMDV (SEQ ID NO:7) wherein X₂ is Y or S and X₄ is S or the amino acid sequence as set forth in SEQ ID NO:8 (DYNYDGN).

According to the present invention, the human monoclonal antibody of the present invention binds to the epitope that consists of the amino acid sequence that ranges from the amino acid residue at position 245 to the amino acid residue at position 275 in SEQ ID NO:1.

In some embodiments, the human monoclonal antibody (“E11”) of the present invention has:
a light chain comprising the complementarity determining regions CDR1L, CDR2L and CDR3L, the CDR1L having the amino acid sequence AGTSSDVGGYYGVS (SEQ ID NO:9) the CDR2L having the amino acid sequence YDSNRPS (SEQ ID NO:10) and the CDR3L having the amino acid sequence SSYTSYSTRV (SEQ ID NO:11) and
a heavy comprising the complementarity determining regions CDR1H, CDR2H and CDR3H, the CDR1H having the amino acid sequence NYYMN (SEQ ID NO:12), the CDR2H having the amino acid sequence GISGSSRYINYADFVKG (SEQ ID NO:13), and the CDR3H having the amino acid sequence SYYSGMDV (SEQ ID NO: 14).

In some embodiments, the human monoclonal antibody (“G12”) of the present invention has:
a light chain comprising the complementarity determining regions CDR1L, CDR2L and CDR3L, the CDR1L having the amino acid sequence AGTSSDVGGYSYVS (SEQ ID NO:15) the CDR2L having the amino acid sequence SDSSRPS (SEQ ID NO:16) and the CDR3L having the amino acid sequence SSYTNSATRV (SEQ ID NO:17) and
a heavy comprising the complementarity determining regions CDR1H, CDR2H and CDR3H, the CDR1H having the amino acid sequence NNYMN (SEQ ID NO:18), the CDR2H having the amino acid sequence NIAGSSRGIYYADFVKG (SEQ ID NO:19), and the CDR3H having the amino acid sequence SSYDYNYDGNGMDV (SEQ ID NO:20).

In some embodiments, the human monoclonal antibody of the present invention comprises a VL domain having at least 90% of identity with SEQ ID NO:21 and/or a VH domain having at least 90% of identity with SEQ ID NO:22.

>E11a. VL having the following structure FR1-CDR1-FR2-CDR2-FR3-CDR3-FR4
SEQ ID NO: 21
QSVLTQPASVSGSPGQSITISCAGTSSDVGGYYGVSWYQQHPGKAPKLMIYYDSNRPSGVSNRFSGSKSGNTASL
TISGLQAEDEADYYCSSYTSYSTRVFGGGTKLAVL
>E11a. VH having the following structure FR1-CDR1-FR2-CDR2-FR3-CDR3-FR4
SEQ ID NO: 22
EVQLVESGGSLVKPGGSLRLSCAASGFTFSNYYMNWVRQAPGKGLEWISGISGSSRYINYADFVKGRFTISRDNA
KNSLYLQMNSLRAEDTAVYYCVRSYYSGMDVWGRGTLVTVSS

In some embodiments, the human monoclonal antibody of the present invention comprises a VL domain having at least 90% of identity with SEQ ID NO:23 and/or a VH domain having at least 90% of identity with SEQ ID NO:24.

>G12a. VL having the following structure FR1-CDR1-FR2-CDR2-FR3-CDR3-FR4
SEQ ID NO: 23
QSVLTQPASVSGSPGQSITISCAGTSSDVGGYSYVSWYQQHPGKAPKLMIYSDSSRPSGVSNRFSGSKSGNTASL
TISGLQAEDEADYYCSSYTNSATRVFGGGTKLAVL
>G12a. VH having the following structure FR1-CDR1-FR2-CDR2-FR3-CDR3-FR4
SEQ ID NO: 24
EVQLVESGGSLVKPGGSLRLSCAASGFTFSNNYMNWVRQAPGKGLEWISNIAGSSRGIYYADFVKGRFTISRDNA
KNSLYLQMNSLRAEDTAVYYCVRSSYDYNYDGNGMDVWGRGTLVTVSS

In some embodiments, the heavy chain and/or the light chain of the antibody of the invention comprises conservative sequence modifications. Thus, one or more amino acid residues within the VL and/or VH domain of the antibody herein disclosed can be replaced with other amino acid residues from the same side chain family and the altered antibody can be tested for binding to ROBO4.

In some embodiments, the antibody of the present invention is an antibody fragment. Fragments can be produced by techniques that are known in the art. For instance, Fab or F(ab′)₂ fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al., Cytokines 16 (3): 106-119 (2001) and Delgado et al., Br. J. Cancer 5 73 (2): 175-182 (1996), the disclosures of which are incorporated herein by reference.

The antibody of the present invention is produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

Accordingly, a further object of the invention relates to a polynucleotide encoding an antibody according to the invention. More particularly the polynucleotide encodes a heavy chain and/or a light chain of an antibody of the present invention.

Typically, said polynucleotide is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. So, a further object of the invention relates to a vector comprising a polynucleotide of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

As used herein, the term “promoter/regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

A further object of the present invention relates to a host cell which has been transfected, infected or transformed by a polynucleotide and/or a vector according to the invention. In some embodiments, the host cell is a non-human embryonic cell.

As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The polynucleotides of the invention may be used to produce an antibody of the present invention in a suitable expression system.

The term “expression system” means a host cell and compatible vector under suitable conditions, e.g., for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, non-human embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant polynucleotide or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Examples of vectors include all those known in the art, including, without limitation, cosmids, plasmids (e.g., naked or contained in liposomes) and viruses (e.g., lentiviruses, retroviruses, adenoviruses, and adeno-associated viruses) that incorporate the recombinant polynucleotide.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

In some embodiments, the antibody (preferably the monoclonal antibody) of the present invention does not comprise a Fc region that mediates antibody-dependent cell-mediated cytotoxicity and thus does not comprise an Fc portion that induces antibody dependent cellular cytotoxicity (ADCC). In some embodiments, the antibody (preferably the monoclonal antibody) of the present invention does not comprise an Fc region that induces complement dependent cytotoxicity (CDC) or antibody-dependent phagocytosis. In some embodiments, the antibody (preferably the monoclonal antibody) of the present invention does not lead, directly or indirectly, to the depletion of cells expressing ROBO4 polypeptides (e.g., do not lead to a 10%, 20%, 50%, 60% or greater elimination or decrease in number of ROBO4⁺ Cells). In some embodiments, the antibody (preferably the monoclonal antibody) of the present invention does not comprise an Fc domain capable of substantially binding to a FcγRIIIA (CD16) polypeptide. In some embodiments, the antibody (preferably the monoclonal antibody) of the present invention lacks an Fc domain (e.g., lacks a CH2 and/or CH3 domain) or comprises an Fc domain of IgG2 or IgG4 isotype. In some embodiments, the antibody (preferably the monoclonal antibody) of the present invention comprises an Fc domain (e.g., of IgG1) with an altered glycosylation profile, resulting in the absence of ADCC activity of the antibody. In some embodiments, the antibody (preferably the monoclonal antibody) of the present invention consists of or comprises a Fab, Fab′, Fab′-SH, F(ab′)₂, Fv, a diabody, single-chain antibody fragment, or a multispecific antibody comprising multiple different antibody fragments. In some embodiments, the antibody (preferably the monoclonal antibody) of the present invention is not linked to a toxic moiety. In some embodiments, one or more amino acids selected from amino acid residues can be replaced with a different amino acid residue such that the antibody has altered C2q binding and/or reduced or abolished CDC. This approach is described in further detail in U.S. Pat. No. 6,194,551 by ldusogie et al.

### Therapeutic Uses:

A further object of the present invention relates to the antibody of the present invention for use as a drug.

More specifically, the present invention provides a method of therapy in a patient in need thereof, comprising administering to the patient a therapeutically effective amount of an antibody of the present invention.

In some embodiments, the antibody of the present invention is particularly the treatment of cancer in a patient in need thereof.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer is not a non-small cell lung carcinoma. In some embodiments, the cancer is not a melanoma.

In some embodiments, the antibody of the present invention is particularly the treatment of bone metastases in a patient in need thereof.

As used herein, the term “bone metastasis” has its general meaning in the art and refers to metastasis of cancer to bone and includes bone metastasis of an arbitrary origin. The term “bone metastasis” preferably includes, but is not limited to, bone metastasis of the cancer described herein, more preferably solid cancer, still more preferably cancer selected from the group consisting of cancers of the breast, the heart, the lung, the small intestine, the large intestine, the spleen, the kidney, the bladder, the head and neck, the ovary, the prostate, the brain, the pancreas, the skin, bone, thymus, the uterine, the testis, the uterine cervix, and/or the liver. According to the present invention, the term “bone metastasis” also preferably includes a bone lesion, preferably an osteolytic and/or osteogenic bone lesion, more preferably an osteolytic bone lesion, still more preferably a bone lesion of myeloma, malignant myeloma, and/or multiple myeloma, particularly an osteolytic bone lesion of myeloma, malignant myeloma, and/or multiple myeloma. According to the present invention, the term “bone metastasis” also preferably includes a bone lesion of Waldenstrom's disease, preferably an osteolytic and/or osteogenic bone lesion of Waldenstrom's disease, more preferably an osteolytic bone lesion of Waldenstrom's disease. The bone metastasis according to the present invention more preferably includes bone metastasis of cancer selected from the group consisting of breast cancer, lung cancer, large intestine cancer, colorectal cancer, kidney cancer, bladder cancer, head and neck cancer, ovary cancer, prostate cancer, brain cancer, pancreatic cancer, skin cancer, thymus cancer, uterine cancer, testis cancer, uterine cervical cancer, and liver cancer.

### Pharmaceutical Compositions:

A further object of the present invention relates to a composition comprising, consisting of or consisting essentially of an antibody of the present invention.

In some embodiments, the composition of the invention is a pharmaceutical composition and further comprises a pharmaceutically acceptable carrier.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g., 1 ml for intramuscular), and between about 1 ng to about 100 mg, e.g., about 50 ng to about 30 mg or more preferably, about 5 mg to about 25 mg, of an antibody of the invention.

The invention will be further illustrated by the following figures and examples. However, these examples and figures should not be interpreted in any way as limiting the scope of the present invention.

### FIGURES

FIG. 1: ELISA on peptides. The biotinylated peptides were coupled to streptavidin magnetic beads. After a saturation step, the various clones to be tested were incubated (in PBS-BSA 2%, 1 hour, 10 μg/ml). After a washing step, the secondary antibody (Sigma A0170) was incubated. After a new washing step (PBS-Tween/PBS), the TMB substrate was added, then the sulfuric acid and the plate was read at 450 nm. In light gray the OD observed on the irrelevant peptide. In dark gray, that observed on the peptide of interest ROBO4. Negative controls are isotype controls specific for other targets.

FIG. 2: Effect of anti-ROBO4 antibodies on the attachment of tumor cells to osteoblastic cells in monolayers. (A) Schematic representation of the protocol. Luciferase-expressing B02 cells (B02-Luc+), pretreated or not pretreated for 1 hour with an anti-ROBO4 antibody (Thermo, G12.3 or E11.3) or an isotype-matched negative control antibody were plated on a MC3T3-E1 osteoblastic cell monolayer. After 18-hour incubation, luciferin was added and tumor cell attachment to osteoblasts was quantified using luciferase activity measurement. Lower left-hand panel: representative photographs of B02 and MC3T3-E1 cell attachment to a plastic surface or B02 cell attachment to a MC3T3-E1 cell monolayer. (B) Effect of a pretreatment of B02 cells with an anti-ROBO4 antibody (10 mg/ml) on tumor cell attachment to MC3T3-E1 osteoblastic cell monolayers, compared to a negative control isotype-matched antibody. Results were expressed as a percentage (%) of the attachment of untreated B02 cells (positive control), set to 100%. Data are the mean±SD of 3 separate experiments. *, **: P<0.05 and 0.01, respectively. ns: non-significant.

FIG. 3: Effect of anti-ROBO4 antibodies on the formation of tumor spheroids. Human parental B02 and KO-ROBO4-B02 breast cancer cells were cultured for 7 days in 3D suspension conditions. Alternatively, B02 cells were pre-treated at day 0 with 10 μg/ml of an anti-ROBO4 antibody (Thermo, G12.3 or E11.3), a negative control antibody or antibodies directed against 13R4 or HER2 (trastuzumab), and subsequently cultured in suspension conditions. Scattered dot plots represent the individual areas of tumor spheroids. Representative images of B02 treated or not treated with an anti-ROBO4 antibody and KO-ROBO4-B02 tumor spheroids are shown. Scattered dot plot data are the median±SD of 1 out of 3 separate experiments. *, **: P<0.05 and 0.01, respectively.

### EXAMPLE

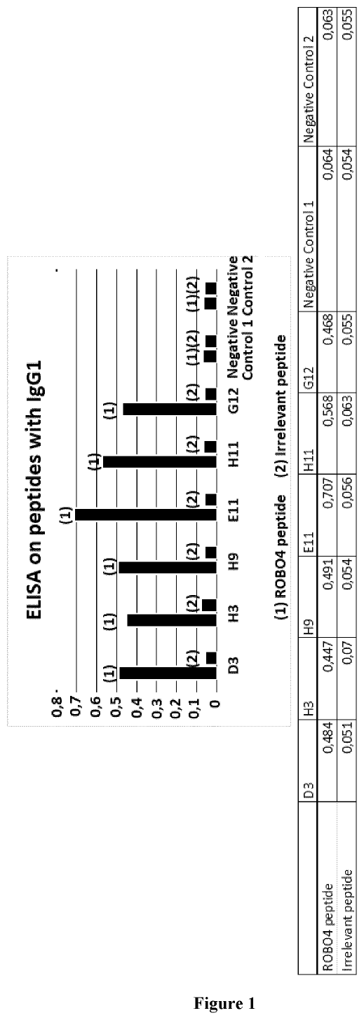

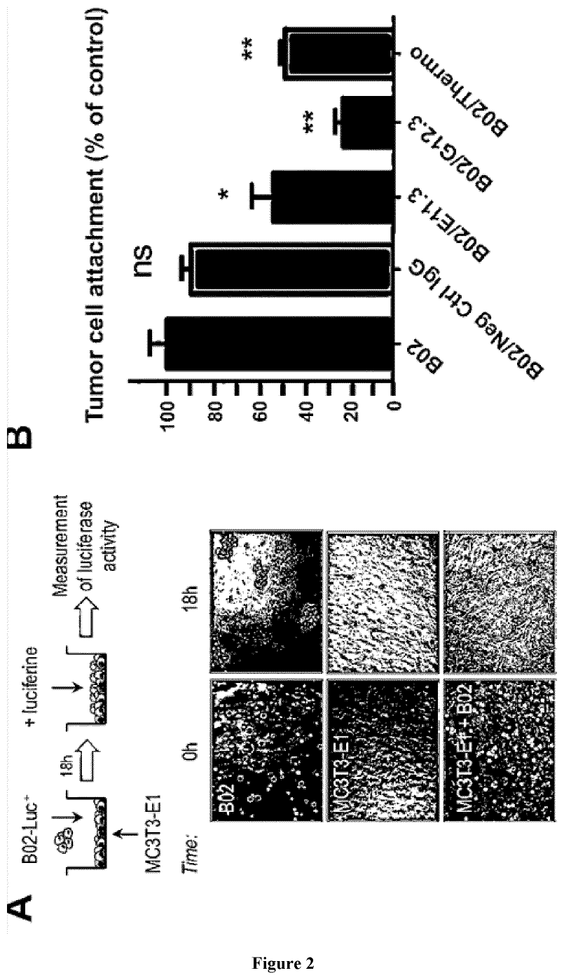

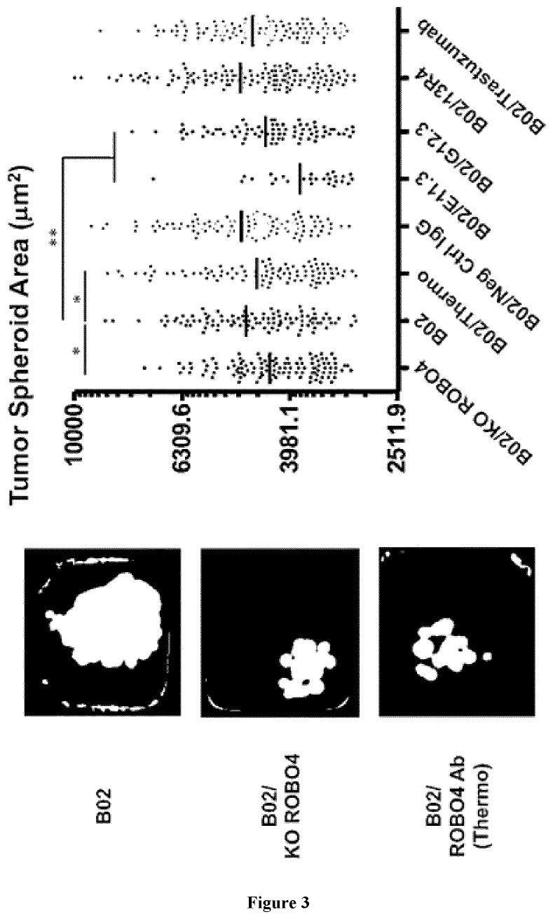

The development of human antibodies directed against ROBO4 were produced by a phage display strategy and the antibody selection was performed by using HEK and HEK stably expressing ROBO4 (HEK-ROBO4) cell lines. As a first step, a batch of 6 different antibodies named D3, H3, H9, E11, H11 and G12 was tested for their binding properties (FIG. 1). The antibodies were then tested for their ability to inhibit the attachment of tumor cells to osteoblastic cells in monolayers (FIG. 2A, FIG. 2B): the G12.3 antibody significantly inhibits said attachment in comparison with the commercial anti-ROBO4 antibody. Finally as shown in FIG. 3 the anti-ROBO4 antibodies E11 and G12 inhibits significantly the formation of the tumor spheroid in comparison with the commercial antibody (MAB24541).

### REFERENCES

Throughout this application, various references describe the state of the art to which this invention pertains. The disclosures of these references are hereby incorporated by reference into the present disclosure.

1. An anti-ROBO4 human monoclonal antibody having:
a light chain comprising the complementarity determining regions CDR1L, CDR2L and CDR3L,
the CDR1L having the amino acid sequence AGTSSDVGGY-X₁₁-X₁₂-VS (SEQ ID NO:2) wherein X₁₁ is Y or S and X₁₂ is G or Y
the CDR2L having the amino acid sequence X₁-DS-X₄-RPS (SEQ ID NO:3) wherein X₁ is Y or S and X₄ is N or S
and the CDR3L having the amino acid sequence SSYT-X₅-X₆-X₇-TRV (SEQ ID NO:4) wherein X₅ is S or N, X₆ is Y or S and X₇ is S or A and
a heavy chain comprising the complementarity determining regions CDR1H, CDR2H and CDR3H,
the CDR1H having the amino acid sequence N-X₂-YMN (SEQ ID NO:5) wherein X₂ is Y or N,
the CDR2H having the amino acid sequence X₁-I-X₃-GSSR-X₈-I-X₁₀-YADFVKG (SEQ ID NO:6) wherein X₁ is G or N, X₃ is S or A, X₈ is Y or G, X₁₀ is N or Y,
and the CDR3H having the amino acid sequence S-X₂-Y-X₄-GMDV (SEQ ID NO:7) wherein X₂ is Y or S and X₄ is S or the amino acid sequence as set forth in SEQ ID NO:8 (DYNYDGN).

2. The anti-ROBO4 human monoclonal antibody of claim 1 that binds to the epitope that consists of the amino acid sequence that ranges from the amino acid residue at position 245 to the amino acid residue at position 275 in SEQ ID NO:1.

3. The anti-ROBO4 human monoclonal antibody of claim 1 that has:
a light chain comprising the complementarity determining regions CDR1L, CDR2L and CDR3L, the CDR1L having the amino acid sequence AGTSSDVGGYYGVS (SEQ ID NO:9) the CDR2L having the amino acid sequence YDSNRPS (SEQ ID NO:10) and the CDR3L having the amino acid sequence SSYTSYSTRV (SEQ ID NO:11) and
a heavy chain comprising the complementarity determining regions CDR1H, CDR2H and CDR3H, the CDR1H having the amino acid sequence NYYMN (SEQ ID NO:12), the CDR2H having the amino acid sequence GISGSSRYINYADFVKG (SEQ ID NO:13), and the CDR3H having the amino acid sequence SYYSGMDV (SEQ ID NO:14).

4. The anti-ROBO4 human monoclonal antibody of claim 1 that has:
a light chain comprising the complementarity determining regions CDR1L, CDR2L and CDR3L, the CDR1L having the amino acid sequence AGTSSDVGGYSYVS (SEQ ID NO:15) the CDR2L having the amino acid sequence SDSSRPS (SEQ ID NO:16) and the CDR3L having the amino acid sequence SSYTNSATRV (SEQ ID NO:17) and
a heavy chain comprising the complementarity determining regions CDR1H, CDR2H and CDR3H, the CDR1H having the amino acid sequence NNYMN (SEQ ID NO:18), the CDR2H having the amino acid sequence NIAGSSRGIYYADFVKG (SEQ ID NO:19), and the CDR3H having the amino acid sequence SSYDYNYDGNGMDV (SEQ ID NO:20).

5. The anti-ROBO4 human monoclonal antibody of claim 1 that comprises a VL domain having at least 90% of identity with SEQ ID NO:21 and/or a VH domain having at least 90% of identity with SEQ ID NO:22.

6. The anti-ROBO4 human monoclonal antibody of claim 1 that comprises a VL domain having at least 90% of identity with SEQ ID NO:23 and/or a VH domain having at least 90% of identity with SEQ ID NO:24.

7. A polynucleotide encoding the anti-ROBO4 human monoclonal antibody of claim 1.

8. The polynucleotide of claim 7 that encodes a heavy chain and/or a light chain of anti-ROBO4 human monoclonal antibody of claim 1.

9. A vector that comprises the polynucleotide of claim 7.

10. A host cell which has been transfected, infected or transformed by the polynucleotide of claim 7 and/or a vector comprising the polynucleotide.

11. (canceled)

12. A method of therapy in a patient in need thereof, comprising administering to the patient a therapeutically effective amount of the anti-ROBO4 human monoclonal antibody of claim 1.

13. A method of treating cancer in a patient in need thereof, comprising administering to the patient a therapeutically effective amount of the anti-ROBO4 human monoclonal antibody of claim 1.

14. A method of treating bone metastases in a patient in need thereof, comprising administering to the patient a therapeutically effective amount of the anti-ROBO4 human monoclonal antibody of claim 1.

15. A pharmaceutical composition comprising the anti-ROBO4 human monoclonal antibody of claim 1 and a pharmaceutically acceptable carrier.


SEQUENCE LISTING (24 sequences)

SEQ ID NO: 1 - protein, 1007 aa, Homo sapiens
MGSGGDSLLGGRGSLPLLLLLIMGGMAQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPEVIATCGVALWLLLLGTAVCIHRRRRARVHLGPGLYRYTSEDAILKHRMDHSDSQWLADTWRSTSGSRDLSSSSSLSSRLGADARDPLDCRRSLLSWDSRSPGVPLLPDTSTFYGSLIAELPSSTPARPSPQVPAVRRLPPQLAQLSSPCSSSDSLCSRRGLSSPRLSLAPAEAWKAKKKQELQHANSSPLLRGSHSLELRACELGNRGSKNLSQSPGAVPQALVAWRALGPKLLSSSNELVTRHLPPAPLFPHETPPTQSQQTQPPVAPQAPSSILLPAAPIPILSPCSPPSPQASSLSGPSPASSRLSSSSLSSLGEDQDSVLTPEEVALCLELSEGEETPRNSVSPMPRAPSPPTTYGYISVPTASEFTDMGRTGGGVGPKGGVLLCPPRPCLTPTPSEGSLANGWGSASEDNAASARASLVSSSDGSFLADAHFARALAVAVDSFGFGLEPREADCVFIDASSPPSPRDEIFLTPNLSLPLWEWRPDWLEDMEVSHTQRLGRGMPPWPPDSQISSQRSQLHCRMPKAGASPVDYS

SEQ ID NO: 2 - protein, 14 aa, synthetic construct
  VARIANT 11: note = X is Y or S
  VARIANT 12: note = X is G or Y
AGTSSDVGGYXXVS

SEQ ID NO: 3 - protein, 7 aa, synthetic construct
  VARIANT 1: note = X is Y or S
  VARIANT 4: note = X is N or S
XDSXRPS

SEQ ID NO: 4 - protein, 10 aa, synthetic construct
  VARIANT 5: note = X is S or N
  VARIANT 6: note = X is Y or S
  VARIANT 7: note = X is S or A
SSYTXXXTRV

SEQ ID NO: 5 - protein, 5 aa, synthetic construct
  VARIANT 2: note = X is Y or N
NXYMN

SEQ ID NO: 6 - protein, 17 aa, synthetic construct
  VARIANT 1: note = X is G or N
  VARIANT 3: note = X is S or A
  VARIANT 8: note = X is Y or G
  VARIANT 10: note = X is N or Y
XIXGSSRXIXYADFVKG

SEQ ID NO: 7 - protein, 8 aa, synthetic construct
  VARIANT 2: note = X is Y or S
  VARIANT 4: note = X is S or DYNYDGN
SXYXGMDV

SEQ ID NO: 8 - protein, 7 aa, synthetic construct
DYNYDGN

SEQ ID NO: 9 - protein, 14 aa, synthetic construct
AGTSSDVGGYYGVS

SEQ ID NO: 10 - protein, 7 aa, synthetic construct
YDSNRPS

SEQ ID NO: 11 - protein, 10 aa, synthetic construct
SSYTSYSTRV

SEQ ID NO: 12 - protein, 5 aa, synthetic construct
NYYMN

SEQ ID NO: 13 - protein, 17 aa, synthetic construct
GISGSSRYINYADFVKG

SEQ ID NO: 14 - protein, 8 aa, synthetic construct
SYYSGMDV

SEQ ID NO: 15 - protein, 14 aa, synthetic construct
AGTSSDVGGYSYVS

SEQ ID NO: 16 - protein, 7 aa, synthetic construct
SDSSRPS

SEQ ID NO: 17 - protein, 10 aa, synthetic construct
SSYTNSATRV

SEQ ID NO: 18 - protein, 5 aa, synthetic construct
NNYMN

SEQ ID NO: 19 - protein, 17 aa, synthetic construct
NIAGSSRGIYYADFVKG

SEQ ID NO: 20 - protein, 14 aa, synthetic construct
SSYDYNYDGNGMDV

SEQ ID NO: 21 - protein, 110 aa, synthetic construct
QSVLTQPASVSGSPGQSITISCAGTSSDVGGYYGVSWYQQHPGKAPKLMIYYDSNRPSGVSNRFSGSKSGNTASLTISGLQAEDEADYYCSSYTSYSTRVFGGGTKLAVL

SEQ ID NO: 22 - protein, 117 aa, synthetic construct
EVQLVESGGSLVKPGGSLRLSCAASGFTFSNYYMNWVRQAPGKGLEWISGISGSSRYINYADFVKGRFTISRDNAKNSLYLQMNSLRAEDTAVYYCVRSYYSGMDVWGRGTLVTVSS

SEQ ID NO: 23 - protein, 110 aa, synthetic construct
QSVLTQPASVSGSPGQSITISCAGTSSDVGGYSYVSWYQQHPGKAPKLMIYSDSSRPSGVSNRFSGSKSGNTASLTISGLQAEDEADYYCSSYTNSATRVFGGGTKLAVL

SEQ ID NO: 24 - protein, 123 aa, synthetic construct
EVQLVESGGSLVKPGGSLRLSCAASGFTFSNNYMNWVRQAPGKGLEWISNIAGSSRGIYYADFVKGRFTISRDNAKNSLYLQMNSLRAEDTAVYYCVRSSYDYNYDGNGMDVWGRGTLVTVSS